(19) United States
(12) Patent Application Publication
(10) Pub. No.: US 2018/0185544 A1
(43) Pub. Date: Jul. 5, 2018

(54) APPARATUS FOR TREATING HUMAN WOUND

(71) Applicant: Ying SHI, San Jose, CA (US)

(72) Inventor: Ying SHI, San Jose, CA (US)

(21) Appl. No.: 15/396,674

(22) Filed: Jan. 1, 2017

(51) Int. Cl. A61L 26/00; A61F 15/00; B65D 75/58; B65D 65/38

(52) U.S. Cl. CPC A61L 26/0057; A61F 15/001; A61L 26/0085; B65D 65/38; B65D 75/5855

(57) ABSTRACT

The invention involves a plaster product for treating burns and scalds. The plaster product includes a piece of flattened biomembrane kept in a sealed sterile plastic envelope or a sealed sterile tinfoil envelope. In use, a regular user or a medical professional opens the top member of the envelope by tearing a designed tip edge from one side to the other such that the biomembrane is entirely exposed, then covers the wound with the biomembrane and discards the bottom member. The biomembrane is the inner membrane of an egg of a bird or an amphibian animal.

### FIELD OF THE INVENTION

The present invention is generally related to a product for over-the-counter use for treatment of certain human wound. More particularly, this invention is related to a plaster for treating burns and scalds using the inner membrane extracted from an egg of a bird or an amphibian animal.

### BACKGROUND OF THE INVENTION

A burn is an injury to the flesh caused by such things as heat, chemicals, friction or electricity. Burns are specifically damage caused to one or multiple layers of skin and flesh by external sources such as heat or chemicals, and range in severity from minor to major. These wounds can range in severity by degrees and, with the exception of first degree burns, most often require medical attention for proper wound assessment and treatment.

First degree burns are also known as superficial burns because the only affect the outermost layer of skin, the epidermis. The skin will become red, dry and painful but eventually the epidermis layer will come off. First degree burns typically don't require medical care.

Second degree burns usually extend to the dermis layer, and can be superficial partial thickness or deep partial thickness in depth. The former affects the epidermis and the dermis, though the damage second layer is superficial. These may be moist and red and could form blisters. Deep partial thickness burns extend fully through the two layers of skin but may be less painful and red than the superficial. However, they take longer to heal and can have more scarring. Second degree burns most likely require medical attention.

Third degree burns extend through the entire dermis and cause the skin to become stiff and turn white or brown in color. They can take months to heal and require burn wound care.

Fourth degree burns extend through the entire skin and as well as the underlying fat, muscle and bone. The skin will turn black and look charred. Fourth degree burns are the most serious and require immediate medical attention and treatment.

In the case of second, third and fourth degree burns, it is crucial to seek immediate medical assistance to avoid infection which can create further complications. Burns, certain scalds, and certain type of wounds that have not been healed for long time, are treated by steps of cleansing the wound surface, sterilizing and dressing. Medical professionals may use wound debridement to remove debris, dead skin and blisters. They will also determine the best wound dressing option. According to the British Medical Journal, hydrocolloid dressings are useful for hands and other small areas with superficial or partial thickness burns. Alginate dressings may also be appropriate as they adhere to the wound. Once the wound heals, the alginate will separate.

For large area burns patients of full thickness skin defects, autologous skin graft is used in burns surgery clinical treatment. Due to large area of skin damage, body fluid effuses from the wound. Since the body fluid includes many types of human proteins, bacteria breed, causing further infection, even in the worst situation causing shock.

However, the existing medicines and treating means are not very effective for many types of human wounds. What is desired is an effective over-the-counter treatment media which is less expensive and easy to use.

### SUMMARY OF THE INVENTION

The invention discloses a plaster product for treating open wounds such as burns and scalds. The plaster product includes a piece of flattened biomembrane kept in a sealed sterile plastic envelope or sterile tinfoil envelope. In use, a regular user or a medical professional opens the top member of the envelope by tearing a designed tip edge from one side to the other such that the biomembrane is entirely exposed, then covers the wound with the biomembrane and discards the bottom member. The biomembrane is the inner membrane of an egg of a bird or an amphibian animal. The membrane extracted from the egg is flattened and preserved in a sterile envelope made of thin, flat, flexible material such as plastic film or tin foil.

The biomembrane has a strong adsorption to human tissue and thus can protect the wound. The biomembrane contains lysozyme which has the function of disinfection and sterilization. More importantly, the biomembrane is a semi permeable, and thus small molecules, such as oxygen and water, can freely enter and exit. This ensures the respiratory function of the wound. Because the large molecules cannot effuse through the biomembrance, the patient will not lose body fluid from the wound. This special structure of the biomembrance can also resist the invasion of the bacteria from the outside. The biomembrane itself and the attached egg white contains a variety of protein and vitamins that can promote wound healing.

Since the shape of biomembrane from animal or bird is spherical, it is difficult to extract and preserve, and therefore it has not been used in clinical practice. This invention fills the gap and makes the biomembrane easy to save and use, so that it can be widely used in daily life and medical field.

### BRIEF DESCRIPTION OF THE DRAWINGS

FIG. 1A is a schematic diagram illustrating the product according to this invention where the top member of the envelope is fixed to the bottom member by a gluing means such as a sterile glue.

FIG. 1B is a schematic diagram illustrating the product according to this invention where the top member is fixed to the bottom member by a stitching means with sealing pressure.

FIG. 2 is a schematic diagram illustrating the product when the top member is torn open showing the biomembrane is exposed before it is applied to the wound.

FIG. 3A is a schematic diagram showing a part of human body having an open wound where the skin is damaged.

FIG. 3B is a schematic diagram showing the product according to the invention is applied to the human body by covering the biomembrane over the wound.

FIG. 4 is a schematic diagram showing the biomembrane is covered over the wound and the envelope for preserving the membrane is discarded.

### DETAILED DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation of the scope of the invention is thereby intended. Any alterations and further implementations of the principles of the invention as described herein are contemplated as would normally occur to one skilled in the art to which the invention relates.

The invention is based on the findings that the inner membrane extracted from an egg of a bird or a poultry or an amphibian animal is beneficial in treating burns, scalds, and many types of human wounds.

In this application, the term biomembrane refers to a biological membrane or a biological film which is typically less than 0.5 mm in thickness.

The typical structure of an egg includes an eggshell, egg white and yolk. Eggshell is made almost entirely of calcium carbonate (CaCO₃) crystals. Bumpy and grainy in texture, an eggshell is covered with as many as 17,000 tiny pores. It is a semipermeable membrane, which means that air and moisture can pass through its pores. The shell also has a thin outermost coating called the bloom or cuticle that helps keep out bacteria and dust.

The egg white is known as the albumen, which comes from albus, the Latin word for “white.” Four alternating layers of thick and thin albumen contain approximately 40 different proteins, the main components of the egg white in addition to water.

The yolk contains less water and more protein than the white, some fat, and most of the vitamins and minerals of the egg. These include iron, vitamin A, vitamin D, phosphorus, calcium, thiamine, and riboflavin. The yolk is also a source of lecithin, an effective emulsifier. Yolk color ranges from just a hint of yellow to a magnificent deep orange, according to the feed and breed of the hen.

Lying between the eggshell and egg white, there are two semi-transparent protein membranes that provide efficient defense against bacterial invasion. The membranes are made partly of keratin, a protein that is also in human hair. The outer membrane sticks to the egg shell while the inner membrane sticks to the egg white. When an egg is first laid, it is warm. As it cools, the contents contract and the inner shell membrane separates from the outer shell membrane to form the air cell. The air cell usually rests between the outer and inner membranes at the egg's larger end. As the egg ages, moisture and carbon dioxide leave through the pores of the shell, air enters to replace them and the air cell becomes larger.

Egg size is usually proportional to the size of the adult bird, from the half gram egg of the bee hummingbird to the 1.5 kg egg of the ostrich. Kiwis have disproportionately large eggs, up to 20% of the female's body weight.

After hundred times of experiments, it is concluded by the inventor that the inner membrane of the egg has an amazing effect in treatment of burns, scalds, and many types of human wounds. The product described below is based on the inventor's findings.

The product for over-the-counter use in treating human wound includes a piece of flattened internal membrane of an egg preserved in a sterile airless envelope made of thin, flat, flexible, water-proof, material such as plastic film or tinfoil. Airless envelope means that the envelope does not include any air. In other words, when the envelope is sealed, the air in the envelope is expelled out.

In the drawings, 10 refers to the thin, flat base member, also bottom member of an envelope or packaging enclosure. 20 refers to the thin, flat top member of the envelope. The top member is attached to the bottom member around the edge such that a space is reserved for content. 30 refers to piece of flattened biomembrane, typically the membrane between the egg shell and egg white. 21 refers to a tip, or an end, a corner of the top member 20, which is not attached or glued to the bottom member for the convenience of peeling off the top member 20 from the bottom member 10. 40 refers to a part of human body. 50 refers to an open wound, such as burn or scald, where the skin is damaged.

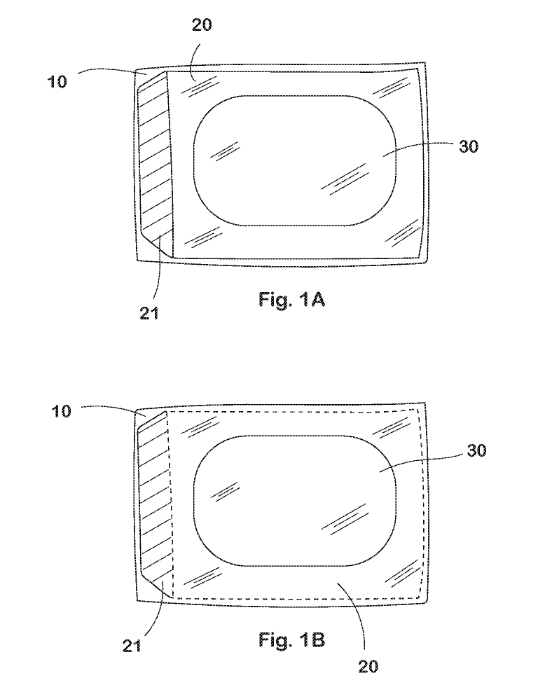

FIG. 1A and FIG. 1B are schematic diagrams illustrating the product according to this invention. In FIG. 1A, the top member 20 is fixed to the bottom member by a gluing means such as a sterile glue for plastics. In FIG. 1B, the top member 20 is fixed to the bottom member by a stitching means with sealing pressure.

FIG. 2 is a schematic diagram illustrating the product when the top member 20 is torn open showing the bio membrane 30 is exposed. Before applying the product to the wound, the user needs first to tear the top member 20. The product is designed in such a way that the top member 20 can be easily torn open by pulling from the edge or tip 21 which is top sealed or attached to the bottom member 10.

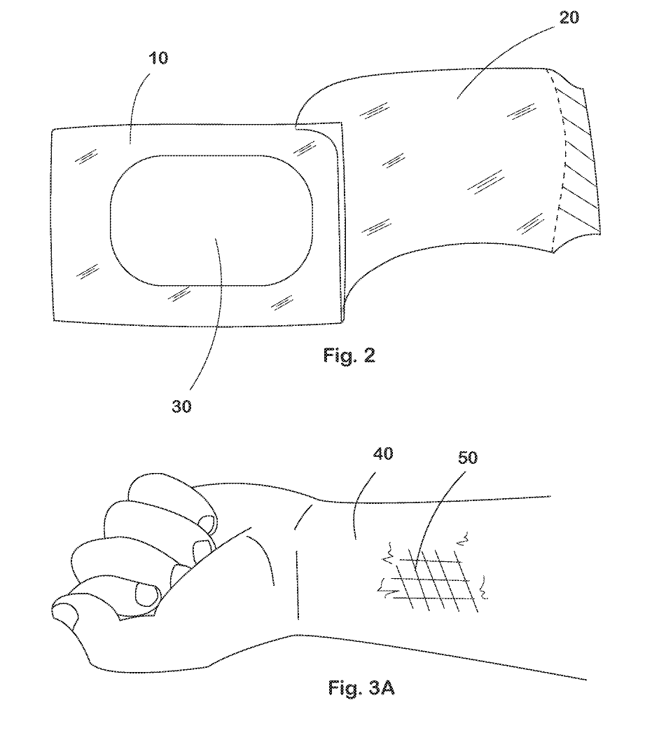

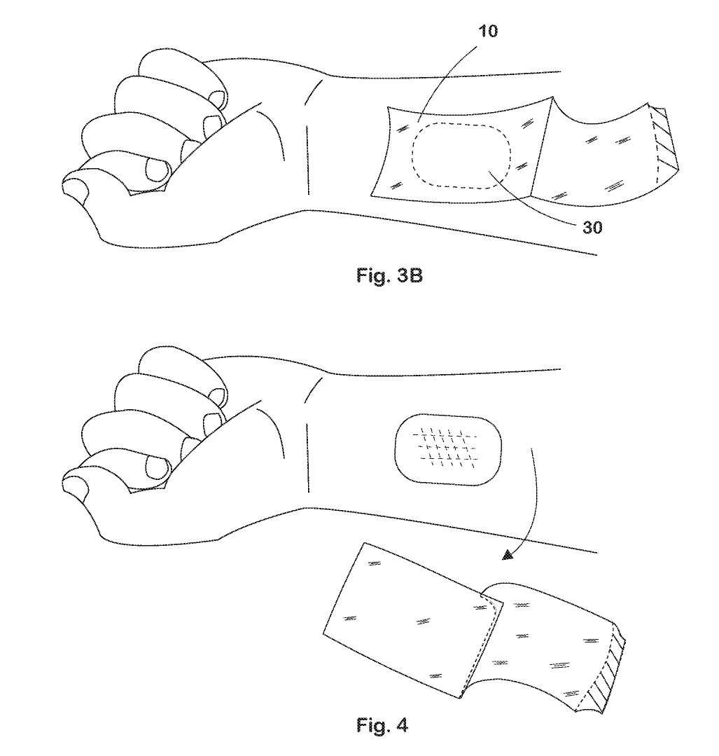

FIG. 3A is a schematic diagram showing a part of human body 40 having an open wound 50 where the skin is damaged. The product according to the invention can be used in various types of open wound where the skin is damaged.

The biomembrane has a strong adsorption to human tissue and thus can well protect the wound. The biomembrane contains lysozyme which has the function of disinfection and sterilization. More importantly, the biomembrane is a semi-permeable. The mall molecules, such as oxygen and water, can freely pass through. This ensures the respiratory function of the wound. Since the large molecules cannot effuse through the biomembrance, the patient will not lose body fluid from the wound. This special structure of the biomembrance can also resist the invasion of the bacteria from the outside. The biomembrane itself and the attached egg white contains a variety of protein and vitamins that can promote wound healing.

After the internal membrane 30 of an egg is covered over the wound 50 for 24-48 hours, the wound 50 will be healed, and new skin will be formed.

FIG. 3B is a schematic diagram showing the product according to the invention is applied to the human body in FIG. 3A by covering the internal membrane 30 over the wound. Note that the internal membrane has two surfaces. The external surface faces to the egg shell. The internal surface faces to the egg white. When the internal membrane 30 is applied to the wound 50, the membrane's internal surface must be gently pasted or covered over the wound 50.

FIG. 4 is a schematic diagram showing the internal membrane 30 is covered over the wound 50 and the envelope for preserving the membrane 30 is discarded.

While one or more embodiments of the present invention have been illustrated above, the skilled artisan will appreciate that modifications and adoptions to those embodiments may be made without departing from the scope and spirit of the present invention.

1. A plaster product for over-the-counter treatment of human wound comprises a piece of flattened internal membrane extracted from an egg of a bird, wherein said membrane is preserved in a sterile airless envelope made of plastic material or tinfoil.

2. The plaster product of claim 1, wherein said envelope comprises a bottom member and a top member attached to said bottom member around outer edges of said members, and wherein said top member can be easily peeled off from said bottom member.

3. The plaster product of claim 2, wherein said top member has at least one tip end being separated from said bottom member for convenience to peel said top member from said bottom member.

4. The plaster product of claim 2, wherein said internal membrane's inner surface faces to said top member, and said internal membrane's outer surface faces to said bottom member.

5. The plaster product of claim 1, wherein internal membrane is immersed in liquid protein.

6. The plaster product of claim 1, wherein internal membrane is immersed in thin albumen of said egg.

7. The plaster product of claim 1, wherein said bird is any of: ostrich, kiwis, goose, chicken and duck.

8. A plaster product for over-the-counter treatment of human wound comprises a piece of flattened internal membrane extracted from an egg of an amphibian animal, wherein said membrane is preserved in a sterile, airless envelope made of plastic material or tinfoil.

9. The plaster product of claim 8, wherein said envelope comprises a bottom member and a top member attached to said bottom member around outer edges of said members, and wherein said top member can be easily peeled off from said bottom member.

10. The plaster product of claim 9, wherein said top member has at least one tip end being separated from said bottom member for convenience to peel said top member from said bottom member.

11. The plaster product of claim 9, wherein said internal membrane's inner surface faces to said top member, and said internal membrane's outer surface faces to said bottom member.

12. The plaster product of claim 8, wherein internal membrane is immersed in liquid protein.

13. The plaster product of claim 8, wherein internal membrane is immersed in thin albumen of said egg.

14. The plaster product of claim 1, wherein said amphibian animal is any of: turtle, salamander, lizard and frog.
